(19) United States
(12) Patent Application Publication
(10) Pub. No.: US 2013/0052280 A1
(43) Pub. Date: Feb. 28, 2013

(54) USE OF THEBROMINE FOR LOWERING CENTRAL BLOOD PRESSURE

(71) Applicants: Richard Draijer, Vlaardingen (NL); Bas Van Den Bogaard, Amsterdam (NL)

(72) Inventors: Richard Draijer, Vlaardingen (NL); Bas Van Den Bogaard, Amsterdam (NL)

(21) Appl. No.: 13/575,339

(22) PCT Filed: Jan. 18, 2011
(86) PCT No.: PCT/EP2011/050574

§ 371 Date: Jul. 26, 2012

(30) Foreign Application Priority Data
Feb. 4, 2010 (EP) 10152665
Apr. 27, 2010 (EP) 10161163
May 19, 2010 (EP) 10163277

(51) Int. Cl. A61K 31/522; A61P 9/12; A61K 33/00; C07D 473/06; A61K 38/14

(52) U.S. Cl. 424/722; 544/273; 514/15.6; 514/263.34

(57) ABSTRACT

The use of theobromine for lowering central blood pressure and/or lowering central pulse pressure in humans and derived benefits, and compositions comprising theobromine.

### FIELD OF THE INVENTION

The present invention relates to the use of theobromine for lowering central blood pressure.

### BACKGROUND OF THE INVENTION

Hypertension is a well-established risk factor for cardiovascular disease. Traditionally, the severity of hypertension has been evaluated by blood pressure (BP) measurements recorded in peripheral arteries, usually the brachial artery. Blood pressure measured conventionally over the brachial artery was assumed to adequately reflect pressures in all parts of the arterial system. It has been reported that this neglected the fact that peripheral (brachial) blood pressure differs from blood pressure recorded in the central arteries, such as the aorta and common carotid artery. It has been shown that central artery/aortic pressure is a stronger predictor of end-organ damage and cardiovascular outcome than conventionally measured brachial pressure (GM London, Medicographia Vol 31, no. 1, 2009 pp 32-37).

Hence, there may be a desire for an agent that specifically targets at lowering central blood pressure (in a clinically relevant amount of at least 2 mm Hg), whether or not peripheral blood pressure (e.g. as measured at the brachial artery) is reduced as well or actually increased. Preferably, such agent should be food grade or have GRAS status, preferably existing in nature, and available in sufficient quantities (either from natural sources or synthetic), and preferably is not a prescription drug.

High central pulse pressure is both an indication for stiffness of the arteries as well as a risk factor on CVD events, and this is especially so for high central pulse pressures (Roman, 2007). Hence, there is a desire for an agent that can reduce central pulse pressures, so as to reduce the risk on CVD and/or kidney problems.

Vlachopoulos et al (American Journal of Cardiology, 99(10), pp 1473-1475, 2007) reports the effects of habitual consumption of cocoa on arterial stiffness and wave reflections, as well as on peripheral and central blood pressure. Antioxidants are referred to as a possible explanation.

Vlachopoulos et al (Sang Thrombose Vaisseaux 19(9), 479-486, 2007) reports effects of nutrition on arterial stiffness and wave reflections. For cocoa and chocolate it is disclosed that such are rich in flavonoids, and that habitual cocoa consumption is inversely associated with blood pressure.

### SUMMARY OF THE INVENTION

It has now surprisingly been found that such objective may be achieved, at least in part, by the use of theobromine, for lowering central blood pressure in humans, and/or for lowering the central pulse pressure in humans and/or for lowering central systolic blood pressure. Hence, in a first aspect the present invention relates to theobromine for use in the treatment of lowering central blood pressure in humans. The invention also relates to a method for lowering central blood pressure in humans by ingestion of theobromine to humans. The invention further relates to a process of using theobromine to lower central blood pressure comprising ingesting an effective amount of theobromine. The invention further relates to the use of theobromine for the manufacture of a medicament or foodstuff for lowering central blood pressure in humans.

In a second aspect the present invention relates to theobromine for use in the treatment of lowering the central pulse pressure in humans. The invention also relates to a method for lowering central pulse pressure in humans by ingestion of theobromine to humans. The invention further relates to a process of using theobromine to lowering central pulse pressure in humans, comprising ingesting an effective amount of theobromine. The invention further relates to the use of theobromine for the manufacture of a medicament or foodstuff for lowering central pulse pressure in humans.

It was found that when humans are given the dimethylxanthine theobromine for ingestion in an effective amount, central blood pressure, e.g. as can be directly measured by an intra-arterial line and high-fidelity pressure transducer in the aorta or estimated by applanation tonometry from the carotid pressure waveform or from the radial pressure waveform (the latter followed by calculation and calibration by using brachial pressure measurements), can be reduced by an amount of at least 2 mmHg, compared to when theobromine is not ingested. This was surprising, as there are several studies in which blood pressure was increased following ingestion of a caffeine-rich beverage as coffee or pure caffeine (a trimethyl xanthine). Such acute and chronic effects are for example published by: Hodgson et al. J Hypertension 17: 457 (1999); Umemura et al. Am J Cardiol 98:1538 (1996); Noordzij et al J Hypertension 23: 921 (2005).

However, in such studies, the blood pressure measured was nearly always peripheral blood pressure (e.g. as measured at the peripheral arteries, usually the brachial artery). Hence, the common understanding was that methylxanthines, when ingested, would raise blood pressure. However, the present inventors have found that such may be true for peripheral BP, but not for central BP, and that central BP in fact can be lowered when an effective amount is given.

As set out above, in following the effects of certain compounds on blood pressure, it is mostly peripheral pressure that is measured. An exception for the trimethylxanthine caffeine, in which central BP is measured, is described by Mahmud and Feely, Hypertension 38: 227 (2001), in which they report an increased central systolic blood pressure (SBP) and increased central diastolic blood pressure (DBP).

### DETAILED DESCRIPTION OF THE INVENTION

“Central blood pressure” is herein to be understood as to refer to the blood pressure at the site of central arteries, such as the aorta and carotid artery. Central blood pressure can suitably be measured non-invasively (as set out below) at the carotis or radialis by applanation tonometry. “Central blood pressure” as used herein thus encompasses aortic blood pressure.

Applanation tonometry makes use of the principle that when a segment of a pipe is flattened, the transmural pressure is equal to the endovascular pressure. A pressure sensor can when positioned on the skin, apply a downward pressure sufficient to flatten a superficial artery, such as the radial artery. Use of radial artery applanation with derivation of a surrogate central aortic pressure waveform by use of a generalized transfer function (TF) was initially reported by Karamanoglu et al. Chen et al. found a good association between this technique and results based on directly recorded central pressure waveforms The use of radial rather than carotid artery applanation to obtain a pressure waveform has been proposed as preferable on the basis that it is an easier technique and more amenable to adequate applanation (Cameron et al. Am J Coll Cardiol 32:1214 (1998); Chen et al Circulation 95:1827 (1997); Karamanoglu et al Eur Heart J 14: 160 (1993); Papaioannou et al. Curr Pharm Des 15(3): 245 (2009).

“Central pulse pressure” is herein to be understood as the difference between central systolic blood pressure (central SBP) and central diastolic blood pressure (central DBP) (the difference of aortic systolic BP and aortic diastolic BP will give the same or similar result).

“Theobromine” herein relates to the molecular structure as set out below, and is chemically known as 2,6-dihydroxy-3,7-dimethylpurine or 3,7-dimethylxanthine (chemical formula: C₇H₈N₄O₂, M_{w}=180.16), including the edible salts thereof.

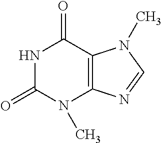

“Tea” for the purposes of the present invention means material from Camellia sinensis var. sinensis and/or Camellia sinensis var. assamica. A beverage will typically comprise at least 85% water, more preferably at least 90%, optimally between 95 and 99.9% by weight of the beverage.

The pH of the beverage may, for example, be from 2.5 to 8, preferably 3 to 6. Most preferably the beverage is tea and comprises tea-derived solids such as catechins, theaflavins, thearubigens and mixtures thereof. The tea solids may, for example, comprise from 0.005 to 3% by weight of the beverage, more preferably from 0.01 to 2%, most preferably from 0.05 to 1% by weight of the beverage.

In a first aspect the present invention relates to theobromine for use in the treatment of lowering central blood pressure in humans. The invention further relates to a method for lowering central blood pressure in humans by ingestion of theobromine to humans. The invention further relates to a process of using theobromine to lower central blood pressure comprising ingesting an effective amount of theobromine. The invention further relates to the use of theobromine for the manufacture of a medicament or foodstuff for lowering central blood pressure in humans.

In a further aspect the present invention relates to theobromine for use in the treatment of lowering the central pulse pressure in humans. The invention further relates to a method for lowering central pulse pressure in humans by ingestion of theobromine to humans. The invention further relates to a process of using theobromine to lower central blood pressure comprising ingesting an effective amount of theobromine. The invention further relates to the use of theobromine for the manufacture of a medicament or foodstuff for lowering the central pulse pressure in humans.

In a further aspect the present invention relates to theobromine for use in the treatment of lowering the central systolic blood pressure in humans. The invention further relates to a method for lowering systolic blood pressure in humans by ingestion of theobromine to humans. The invention further relates to a process of using theobromine to lower the central systolic blood pressure in humans comprising ingesting an effective amount of theobromine.

The invention further relates to the use of theobromine for the manufacture of a medicament or foodstuff for lowering central pulse pressure in humans.

Herein, lowering is preferably at least 2 mm Hg, more preferably at least 3 mmHg.

Therapeutic regimens that preferentially decrease central BP are better associated with organ protection and improved cardiovascular outcomes (than peripheral BP). This was observed in a cross-sectional and longitudinal study in which central BP as well as peripheral BP was associated with left ventricular mass, carotid intima-media thickness, estimated glomerular filtration rate, and 10-year all-cause and cardiovascular mortality in 1272 participants. Specifically central systolic BP predicted cardiovascular mortality (Wang et al. J Hypertens 27: 461-467 (2009).

A reduction in central blood pressure may prevent carotid arterial remodeling, which occurs at increased pressure to reduce wall stress, thus avoiding intima-media thickening. Hence, the present invention further relates to theobromine for use in the treatments of reducing thickening of the arteries, for organ protection and for reducing cardiovascular mortality. The invention also relates to a method for reducing thickening of the arteries, for organ protection and for reducing cardiovascular mortality by ingestion of theobromine. The invention further relates to a process of using theobromine to reduce thickening of the arteries, for organ protection and for reducing cardiovascular mortality in humans comprising ingesting an effective amount of theobromine. The invention further relates to the use of theobromine for the manufacture of a medicament or foodstuff for reducing thickening of the arteries, for organ protection and for reducing cardiovascular mortality.

Also, microvascular damage in the brain and kidneys may be limited by lowering central blood pressure, because these organs have a low resistance to flow, and are extremely vulnerable to high-pressure fluctuations that occur in carotid and renal arteries. Highly pulsatile pressure and flow may predispose to renal dysfunction and stroke (Laurent & Cockroft, isbn 978-2-84299-943-8). Hence, the invention further relates to theobromine for use in the treatment of reducing the risk on renal dysfunction and/or for use in the treatment of reducing renal dysfunction. The invention further relates to a process of using theobromine to reduce thickening of the arteries, for organ protection and for reducing cardiovascular mortality in humans comprising ingesting an effective amount of theobromine. The invention also relates to a method for reducing the risk on renal dysfunction and/or reducing renal dysfunction by ingestion of theobromine.

Thus, the invention further relates to theobromine for use in the treatment of lowering microvascular damage to the brain and/or kidney. The invention also relates to a method for lowering microvascular damage to the brain and/or kidney by ingestion of theobromine. The invention further relates to a process of using theobromine to reduce microvascular damage to the brain and/or kidney in humans comprising ingesting an effective amount of theobromine.

Also, in view of the above, the invention further relates to theobromine for use in the treatment of reducing the burden on the heart. The invention also relates to a method for reducing the risk on stroke by ingestion of theobromine. The invention further relates to a process of using theobromine to reduce the burden on the heart in humans comprising ingesting an effective amount of theobromine.

The invention further relates to theobromine for use in the treatment of prevention or treatment of kidney perfusion dysfunction, chronic kidney disease or end-stage renal disease.

a method for the prevention or treatment of kidney perfusion dysfunction, chronic kidney disease or end-stage renal disease in humans by ingestion of theobromine. The invention further relates to the use of theobromine in the manufacture of a food or medicament for the prevention or treatment of kidney perfusion dysfunction, chronic kidney disease or end-stage renal disease in humans, and also the use of theobromine in the manufacture of a food or medicament for lowering microvascular damage to the brain and/or kidney, and/or for protecting against renal dysfunction and/or stroke and/or for reducing the risk on renal dysfunction and/or stroke.

It is believed by many experts in the field that lowering the level central blood pressure of humans may result in a reduced risk on cardiovascular disease (CVD). Without wishing to be bound by theory, as a possible explanation for this: the central artery BP—and not brachial BP—is directly “facing” the left ventricle. As such, it is the central BP and/or pulse pressure (pressure difference between SBP and DBP), and not peripheral BP, that constitutes the afterload placed on the ventricle during cardiac ejection. As a consequence, central systolic pressure is more strongly associated with left ventricular mass and function, and aortic systolic and pulse pressure are much stronger predictors of clinical outcomes than conventional brachial pressure. Thus, the invention further relates to theobromine for use in the treatment of lowering the risk on cardiovascular disease through lowering of central blood pressure and/or through lowering central pulse pressure. The invention further relates to a method for lowering the risk on cardiovascular disease through lowering of central blood pressure and/or through lowering central pulse pressure. Also, following the above, the invention further relates to theobromine for use in the treatment of reducing the blood pressure that the left ventricle of the heart experiences, as well as for use in the treatment of left ventricle hypertrophy in humans. The invention further relates to a method for reducing the blood pressure that the left ventricle of the heart experiences, as well as to a method for treating left ventricle hypertrophy in humans, by ingestion of theobromine. The invention further relates to the use of theobromine in the manufacture of a food or medicament to reduce the blood pressure that the left ventricle of the heart experiences, and the invention also relates to the use of theobromine in the manufacture of a food or medicament for the prevention or treatment of left ventricle hypertrophy in humans.

In a further aspect the present invention relates to theobromine for use in the treatment of lowering the risk on cardiovascular disease through lowering of central blood pressure and/or through lowering central pulse pressure. The invention further relates to a method for lowering the risk on cardiovascular disease through lowering of central blood pressure and/or through lowering central pulse pressure, by ingestion of theobromine. The invention further relates to the use of theobromine for the manufacture of a medicament or foodstuff for lowering the risk on cardiovascular disease through lowering of central blood pressure and/or through lowering central pulse pressure. Cardiovascular disease in this connection comprises pathophysiological effects to blood vessels and/or organs of increased central blood pressure. Also, reducing the central systolic and/or pulse pressure will reduce the burden on the heart, which exists with high central blood pressures. Hence, in a further aspect of the invention it relates to theobromine for use in the treatment of reducing the burden on the heart. Likewise, the invention further relates to a method for reducing the burden on the heart by ingestion of theobromine. The invention further relates to the use of theobromine for the manufacture of a medicament or foodstuff for reducing the burden on the heart.

For the desired effect of theobromine for use in the treatments herein described, theobromine will need to be ingested (preferably orally). Thus, the invention relates to theobromine for use in the treatments as set out above for the given benefits, by ingestion of theobromine or a composition comprising theobromine.

Not all methylxanthines are expected to display the effect found for theobromine and/or in a similar magnitude. For theobromine, it was found that with a single daily dose of 1000 mg theobromine, central BP could be reduced by 3.3% (p=0.003). For actual use, the required amount of theobromine that is required for a desired reduction in central BP can easily be determined by a skilled person without undue burden. A clinical trial along the lines as set out in the example herein can be used to determine such amount. Hence, the invention further relates to theobromine for use in the treatments as set out herein, wherein the theobromine is ingested in an effective amount to achieve a lowering of central blood pressure and/or central pulse pressure by at least 2%, more preferably by at least 3%.

The effect of central BP lowering can already be achieved after several weeks of ingesting the active here concerned, and can last for more than one hour up to 8 hours or more after the last dose.

As set out above, the effective amount or desired amount of theobromine will depend on e.g. the desired lowering of central BP and/or central pulse pressure. However, when balancing efficacy, safety, cost and/or ease of application it is preferred that for the theobromine for use in the treatments according to the present invention, the treatment comprises ingestion of theobromine in an amount of from 300 to 2000 mg theobromine per day. More preferably, when balancing efficacy, safety, cost and/or ease of application it is preferred that the theobromine for use in the treatments according to the present invention, the treatment comprises ingestion of theobromine in an amount of from 400 to 1800 mg theobromine per day, even more preferably from 500 to 1500 theobromine mg per day, yet more preferably from 600 to 1400 mg theobromine per day, more preferably from 700 to 1300 mg theobromine per day, most preferably from 750 to 1250 mg theobromine per day. These are also the preferred amounts for the methods as described herein.

It was found by the present inventors that heart rate and peripheral BP can be elevated for some time by ingestion of the effective amount of theobromine. It is believed that this can be reduced (if desired) when the use of the present invention of ingesting theobromine is occurring jointly with food, or some time after the consumption of food. Hence, it may be preferred for the theobromine for use in the treatments according to this invention for the given benefits that theobromine is ingested jointly with or less than 1, more preferably less than 0.5 hour after a meal or snack of at least 100 kcalories, preferably containing at least 3 grams of fat.

It is believed by many experts in the field that lowering the central blood pressure of humans may result in a reduced risk of cardiovascular disease (CVD). As a possible explanation for this: the central artery BP—and not brachial BP—is directly “facing” the left ventricle, it more realistically reflects cardiac load. Aortic systolic pressure is more strongly associated with left ventricular mass and function, and aortic systolic and pulse pressure are much stronger predictors of clinical outcomes than conventional brachial pressure. Following this, the invention further relates to theobromine for use in the treatment of left ventricle hypertrophy in humans.

As it is believed that an elevated central BP is detrimental in the long run to organs, such as the kidneys, it is believed that the ingestion of theobromine may prove beneficial against reduced kidney perfusion, chronic kidney disease or end-stage renal disease in humans. Hence, in another aspect the invention further relates to theobromine for use the treatment of chronic kidney disease or end-stage renal disease in humans.

Also, for theobromine for use in the treatments as set out herein, when balancing efficacy, safety, cost and/or ease of application it may be preferred that in such uses and treatments, it relates to ingestion of from 300 to 2000 mg theobromine per day, more preferably 400 to 1800 mg theobromine per day, even more preferably from 500 to 1500 theobromine mg per day, more preferably from yet more preferably 600 to 1400 mg theobromine per day, yet even more preferably from 700 to 1300 mg theobromine per day, most preferably from 750 to 1250 mg theobromine per day.

For the uses and treatments as set out herein and for the desired effects, it is preferred (as it is believed that the effects take some time to occur after first ingestion) that theobromine is ingested for a dosing regime of at least 1 dosing per day on 10 days in a 2 week period, or at least 1 dosing per day on 15 days in a 3 week period, preferably at least 14 daily dosings in 2 weeks, even more preferably at least 21 daily dosings in 3 weeks.

In another aspect of the present invention, it relates to an edible composition comprising per daily serving size theobromine in an amount effective for reducing central blood pressure and/or central pulse pressure with at least 2 mmHg, wherein the theobromine is in a slow release format providing release of theobromine over at least 4 hours after ingestion, and preventing large peak values as measured in blood.

It is believed that the concurrent (with the lowered central BP) elevated heart rate and/or elevated peripheral BP can be reduced when the theobromine has a fairly gradual uptake in the blood, reaching it Tₘₐₓ 2-4 h instead 0.5-1 h after ingestion. Hence, it may be preferred that the composition as set out herein has the format of one or more slowly digestible capsules, pills, tables or granules (all comprising the theobromine). Most preferred are slow-release capsules. Such capsules are well known in the art, and provide the additional benefit that the bitterness of theobromine is not or less noticed by the consumer taking such, and also it is compatible with the effective dosing amounts.

The edible compositions as set out herein preferably comprises (per dosage for a day) theobromine in an amount of from 300 to 2000 mg, preferably from 400 to 1800 mg theobromine, more preferably from 500 to 1500 theobromine mg, even more preferably from 600 to 1400 mg theobromine, yet even more preferably from 700 to 1300 mg theobromine, most preferably from 750 to 1250 mg theobromine, including any combination of a lower and upper range herein. Preferably, the compositions as set out herein are not frozen confection.

It is believed that a more gradual uptake of theobromine can also be achieved when it is ingested together with a certain amount of fat or oil. Hence, as an alternative to the capsules, pills, or tablets, the edible composition as set out herein may comprise fat and theobromine, wherein the amount of theobromine on fat is at least 1% by weight, more preferably at least 2% by weight, most preferably at least 4% by weight, wherein it is desired that the minimum amount of fat is at least 5 g, preferably 10 g. Preferably, the amount of theobromine is such that it is 300 to 2000 mg per dosing, more preferably from 400 to 1800 mg theobromine, yet more preferably from 500 to 1500 theobromine mg, even more preferably from 600 to 1400 mg theobromine, yet even more preferably from 700 to 1300 mg theobromine, most preferably from 750 to 1250 mg theobromine, including any combination of a lower and upper range herein. The concentration of theobromine in pills, tablets or capsules is preferably at least 30% by weight on the pill, tablet or capsule, more preferably at least 50% by weight, even more preferably at least 70% by weight.

Theobromine as herein referred to may be combined with known agents for reducing central blood pressure and/or central pulse pressure, such as potassium (e.g. in daily dosings of 200 to 400 mg, preferably at least 300 mg) and/or lactotripeptides as such combined approach may reduce the central blood pressure even further. Hence, the invention further relates to an edible composition comprising theobromine and potassium, wherein the weight ratio theobromine:potassium is from 1:1 to 20:1, preferably from 2:1 to 6:1. Likewise, the invention also relates to an edible composition comprising theobromine and lactotripeptides, wherein the weight ratio theobromine:lactotripeptides is from 60:1 to 400:1, preferably from 100:1 to 300:1. Preferred lactotripeptides herein are Val-Pro-Pro and Ile-Pro-Pro.

Suitable vehicles among foodstuffs for theobromine according to the present invention include tea-based beverages (including rehydratable precursurs of tea, e.g. instant tea). Hence, the invention further relates to a beverage comprising tea and theobromine, wherein the amount theobromine is at least 0.02% by weight of the total beverage, preferably at least 0.03% by weight, most preferably at least 0.04% by weight.

Likewise, the invention further relates to a rehydratable beverage composition having a moisture content of below 15% by weight, and which comprises theobromine in an amount of from 10 to 80% by weight based on the total composition, and tea solids in a weight ratio theobromine:tea solids of 10:1 and 1:5.

In the use and compositions as set out herein, it is preferred that, if next to theobromine cocoa polyphenols are present in the compositions, the amount of such cocoa polyphenols is limited (e.g. for colour reasons) to less than 3 times (preferably less than 2 times) the amount of theobromine. In other words, if the amount of theobromine is 1000 mg in a composition or dosing, it is preferred that the amount of cocoa polyphenols is below 3000 mg (preferably below 2000 mg) in the same composition or dosing. Also, it is preferred that the amount of cocoa polyphenols is limited to an amount of below 20% by weight on the total composition.

In the present invention, the compositions are preferably substantially free of cyclooxygenase-2 selective inhibitor or isomer, pharmaceutically acceptable salt, ester, or prodrug thereof, e.g. as such are less suitable to be part of compositions offered as foodstuffs or functional foods.

### Examples

In a randomised controlled cross-over study with 42 untreated prehypertensive males and females the effect of cocoa-containing drinks either high in polyphenols or high in polyphenols and theobromine was assessed on blood pressure (BP).

The subjects consumed for 3 weeks daily (in the morning before breakfast) 200 ml acidified dairy drink as in table 1, with added to that 1) none (control drink), 2) 3.65 g cocoa powder (Acticoa, Barry-Callebaut) containing 500 mg polyphenols and 79 mg theobromine (amount in product specification Acticoa) and 3) 5 g cocoa powder containing 500 mg polyphenols and 79 mg theobromine plus added thereto 918 mg pure theobromine (Fagron, Ph. Eur. 5.7) (in total about 1 g theobromine, when the naturally present 79 mg theobromine is added up to the additional 918 mg theobromine).

TABLE 1
composition of the dairy drink (wt % of major components)¹

| | placebo | cocoa | cocoa + theobromine |
|---|---|---|---|
| skimmed milk | 30.5 | 30.5 | 30.5 |
| cream | 0.97 | 0.97 | 0.97 |
| demi water | 62.9 | 61.5 | 58.7 |
| cocoa powder (Acticoa) | 0 | 1.82 | 1.82 |
| theobromine in NaOH² | 0 | 0 | 2.7 |
| sucrose | 3 | 3 | 3 |
| lactic acid | 0.4 | 0.4 | 0.4 |
| flavours | 0.55 | 0 | 0 |
| colourance | 0.09 | 0.08 | 0.07 |
| total | 98.38 | 98.27 | 98.16 |

¹200 g dairy product was consumed daily.
²Pure theobromine powder was dissolved in 1M NaOH to a stock concentration of approximately 170 mg theobromine per ml. This solution was diluted 38.5 times with the dairy drink (1 part stock solution + 37.5 parts drink).

Inclusion criteria: prehypertensive or grade I hypertensive subjects, age 40-70, with ≦2 additional cardiovascular risk factors, not on active anti-hypertensive treatment. Details are set out in table 2.

TABLE 2

| Parameters | | Mean | SD | 95% CI |
|---|---|---|---|---|
| n | | 42 | | |
| Age | yr | 62 | 4.5 | 60.7-63.5 |
| Male | no (%) | 32 (76%) | | |
| Office SBP | mmHg | 142 | 14.0 | 137-146 |
| Office DBP | mmHg | 84 | 7.9 | 82-87 |
| Length | cm | 177 | 8.1 | 175-180 |
| Weight | kg | 82 | 9.0 | 79-85 |
| BMI | kg/m² | 25.9 | 2.4 | 25.2-26.7 |
| Fasting | mmol/L | 4.9 | 0.6 | 4.7-5.1 |
| glucose | | | | |
| TC | mmol/L | 5.77 | 0.77 | 5.52-6.01 |
| LDL-C | mmol/L | 3.72 | 0.66 | 3.52-3.93 |
| HDL-C | mmol/L | 1.55 | 0.42 | 1.42-1.68 |
| Triglycerides | mmol/L | 1.06 | 0.41 | 0.93-1.19 |
| Smoking | no (%) | 1 (2%) | | |

Peripheral BP was measured by 24-hours ambulatory BP at the brachial artery, while central blood pressure was measured by applanation tonometry at the radial artery, using a transfer function to estimate central pulse wave forms, systolic and diastolic BP (SBP and DBP, respectively), pulse pressure (SBP-DBP) and augmentation index. The measurements were done at baseline and after each 3 weeks intervention period, which were separated by 2 weeks washout periods.

Inclusion criteria: prehypertensive or grade I hypertensive subjects, age 40-70, with ≦2 additional cardiovascular risk factors, not on active anti-hypertensive treatment.

### Methodology:

### Pulse Wave Analysis

Radial artery pressure waveforms of the non-dominant arm were sampled over 10 seconds with a Millar tonometer (SPC-301, Millar Instruments). The mean arterial pressure and diastolic BP of the radial pressure waveform were used to calibrate carotid measurements as described by Laurent et al. (Eur Heart J 27(21): 2588 (2006)). Calibration of the artery tonometer pressure wave is based on the observation that mean BP is constant throughout the large artery tree and that diastolic BP does not change substantially. Waveforms were processed using dedicated software (SphygmoCor version 8, AtCor). The integral system software was used to calculate an averaged radial artery waveform. The radial pressure waveform was used to derive a corresponding central aortic pressure waveform using a previously validated generalized transfer function (Chen et al. Circulation 95(7): 1827 (1997); Pauca et al. Hypertension 38(4): 932 (2001)). Aortic pressure waveforms were subjected to further analysis by the SphygmoCor software to identify the time to the peak/shoulder of the first and second pressure wave components (T1, T2) during systole. The pressure at the peak/shoulder of the first component is identified as P1 height (outgoing pressure wave), and the pressure difference between this point and the maximal pressure during systole (ΔP or augmentation) was identified as the reflected pressure wave occurring during systole. Augmentation index (AIx), defined as the ratio of augmentation to central pulse pressure, is expressed as a percentage: AIx=(ΔP/PP)×100, where P is pressure and PP is pulse pressure. AIx is a measure of arterial stiffness and an independent predictor of cardiovascular disease. Pulse pressure amplification (PPA) is expressed as the ratio of central pulse pressure (CPP) to peripheral (brachial) pulse pressure (PPP): PPA=PPP/CPP. The pulse wave consists of an incident wave generated by the left ventricular ejection and of a reflecting wave reflecting from peripheral vasculature. Pulse waves travel faster in stiffer arteries, the subsequent early return of the reflecting wave in stiff arteries will increase or augment central aortic pulse pressure. This phenomenon can be expressed in AIx or PPA.

At least 2 consecutive radial pressure wave samplings were recorded for each patient visit as described above, and data from the mean of the resulting central aortic pressure waveforms were recorded. The investigator was blinded for the patients' characteristics and treatment.

### 24-Hour Ambulatory Blood Pressure Measurements

An automatic ambulatory BP monitor (Spacelabs 90207, Spacelabs In., Redmond, Wash., USA) was placed on the non-dominant arm in all subjects. Systolic and diastolic BP and heart rate (HR) were recorded over at least 24 h. The ambulatory BP monitor was programmed to record BP every 15 min during the day (07:00-23:00 h) and every 30 min at night (23:00-07:00 h). The following predefined day and night periods were used for analysis: day 09:00-21:00 and night 12:00-6:00. Mean 24-hours, daytime and night-time ambulatory SBP, DBP and HR were reported.

### Descriptive Statistics

Baseline data was expressed as mean plus standard deviation for variables with a parametric distribution and median plus range for variables with a skewed distribution. Categorical data was calculated using chi-square statistics. Continuous data was calculated using one-way ANOVA with correction for baseline characteristics where appropriate. A 2 sided p-value <0.05 is considered significant.

### Results

85 subjects were screened and 42 of them were found eligible to enter the study. The population characteristics at baseline were as followed (average values with between brackets standard deviation): 32 males (76%), age 62 (4.5), office SBP 142 (14) and DBP 84 (8), BMI 25.9 (2.4), total cholesterol 5.77 (0.77), 2% smokers.

Compliance was >99% as estimated from counting returned empty bottles.

### Ambulatory-BP Results (Per Protocol Population, Corrected for Baseline Values; Between Brackets Standard Error of Mean):

24 h-SBP/Daytime SBP/Nighttime SBP
control: 123.9 (1.0)/129.8 (1.2)/112.5 (1.3)
cocoa 125.4 (1.0)/131.3 (1.2)/114.2 (1.3)
cocoa+theobromine 127.1^{a} (1.0)/133.5^{a} (1.2)/115.1 (1.3)
^{a}significantly different from control, p<0.05
24 h-DBP/Daytime DBP/Nighttime DBP
control: 76.4 (0.7)/80.5 (0.7)/68.6 (0.9)
cocoa: 77.7^{b} (0.7)/81.7 (0.7)/69.9 (0.9)
cocoa+theobromine: 77.8^{b} (0.7)/82.3^{b} (0.7)/69.3 (0.9)
^{b}significantly different from control, p<0.05
24 h-HR/Daytime HR/Nighttime HR
control: 66.9 (0.8)/71.1 (1.1)/60.4 (0.8)
cocoa: 67.4 (0.8)/71.9 (1.1)/60.7 (0.8)
cocoa+theobromine: 70.9^{c} (0.8)/76.1^{c} (1.1)/63.3^{c} (0.8)
^{c}significantly different from control, p<0.05

### Central BP and Pulse Pressure Results (per Protocol Population, Corrected for Baseline Values; Between Brackets Standard Error of Mean):

### Central SBP/Central DBP/AIx

control: 128.6 (1.5)/82.6 (0.9)/19.8 (0.7)
cocoa: 129.5 (1.5)/82.8 (0.9)/19.4 (0.7)
cocoa+theobromine: 124.3^{d}(1.5)/81.6 (0.9)/13.2^{d}(0.7)
^{d}significantly different from control, p<0.05

Consumption of a dairy drink with cocoa powder and added theobromine (1 g in total) decreased central SBP by 4.3 and 5.2 mmHg compared to a matched control dairy drink or a dairy drink with cocoa powder alone, respectively.

Consumption of a dairy drink with cocoa powder and added theobromine (1 g in total) tended to decrease central DBP by 1.0 and 1.2 mmHg compared to a matched control dairy drink or a dairy drink with cocoa powder alone, respectively.

Central pulse pressure (SBP-DBP) decreased from 46 (control) and 46.7 (cocoa) to 42.7 mmHg (cocoa+theobromine).

Consumption of a dairy drink with cocoa powder and added theobromine (1 g in total) decreased augmentation index by 6.4 and 6.2% compared to a matched control dairy drink or a dairy drink with cocoa powder alone, respectively.

In contrast, peripheral SBP and HR were increased after consumption of a dairy drink with cocoa and theobromine compared to control and cocoa alone.

In conclusion: Central systolic BP and central pulse pressure as well as augmentation index were decreased after consumption of a drink with cocoa powder and theobromine. The effect is attributed solely to the added theobromine, because a drink with cocoa alone did not exert any decrease in these parameters compared to a control drink. Central systolic BP, central PP and AIx are all indicators of the pressure burden on heart, brain and kidney. Theobromine therefore can contribute to function preservation or improvement of these organs.

1-12. (canceled)

13. Edible composition comprising per daily serving size theobromine in an amount effective for reducing central blood pressure and/or aortic pulse pressure with at least 2 mmHg, which theobromine is in a slow release, format so that Tₘₐₓ is reached from 2 to 4 hours after ingestion.

14. Edible composition according to claim 13, wherein the composition has the format of one or more slowly digestible capsules, pills, tablets or granules, such that Tₘₐₓ is reached from 2 to 4 hours after ingestion.

15. Edible composition according to claim 13, further comprising potassium, wherein the weight ratio theobromine:potassium is from 1:1 to 20:1.

16. Edible composition according to claim 13, further comprising lactotripeptides, wherein the weight ratio theobromine:lactotripeptides is from 60:1 to 400:1, preferably from 100:1 to 300:1.

17. Beverage comprising tea and theobromine, wherein the amount theobromine is at least 0.02% by weight of the total beverage.

18. Rehydratable beverage composition having a moisture content of below 15% by weight, and which comprises theobromine in an amount of from 10 to 80% by weight based on the total composition, and tea solids in a weight ratio theobromine:tea solids of 10:1 and 1:5.
